(19) United States
(12) Patent Application Publication
(10) Pub. No.: US 2026/0257218 A1
(43) Pub. Date: Sep. 3, 2026

(54) LASER WELDED ENCLOSURE FOR ELECTRONICS, CIRCUITRY OR SENSORS

(71) Applicants: SCHOTT AG, Mainz (DE); SCHOTT PRIMOCELER OY, Tampere (FI)

(72) Inventors: Jens Ullrich THOMAS, Mainz (DE); Antti MÄÄTTÄNEN, Tampere (FI); Christian OTT, Landshut (DE)

(73) Assignees: SCHOTT AG, Mainz (DE); SCHOTT PRIMOCELER OY, Tampere (FI)

(21) Appl. No.: 18/878,719

(22) PCT Filed: Sep. 28, 2023
(86) PCT No.: PCT/EP2023/076927

§ 371 Date: Dec. 24, 2024

(30) Foreign Application Priority Data
Sep. 29, 2022 (EP) 22198679.7

(51) Int. Cl. B01L 3/00

(52) U.S. Cl. CPC B01L 3/502715; B01L 3/502707; B01L 3/502738; B01L 3/502746; B01L 2300/049; B01L 2300/0636

(57) ABSTRACT

An enclosure is provided having a first and a second substrate, a common laser weld zone, a function zone enclosed inside the enclosure, and a fluid channel allowing for a fluid flow between the function zone and an outside area. The first substrate is transparent at least in part and/or at least for a wavelength bandwidth. The common laser weld zone permanently joins the first substrate to the second substrate and/or to provide for a hermetic sealing between the first substrate and the second substrate. The function zone includes a cavity and is inside the enclosure.

### FIELD OF THE INVENTION

The invention is related to an enclosure, for example, for providing an at least partially hermetically sealed compartment or cavity in at least two layers of substrates, an immersible sensor element, an immersible donator element and an immersible specific filter element.

### BACKGROUND AND SUMMARY OF THE INVENTION

Enclosures can be used, for example, to protect electronics, circuitry or sensors. It is possible to use hermetically sealed implementations of aforementioned enclosures for medical implants, for example, in a therapy to cure a heart disease, or, for example, in a retina or for any type of bio-processor. Known are bio-processors, which are made from titan. It is principally known to provide several parts or layers and to arrange them such that in an inner region components can be situated. For example European Patent EP 3 012 059 B1 shows a method for manufacturing a transparent component for protecting an optical component. A new laser welding method is used therein.

In some use cases that serve as one of several problems to be solved by the present invention it is desirable to measure or influence one or several properties of the fluid or material surrounding the enclosure. Sensors can be used or implanted by means of the invention, for example, for use in particular rough or (chemically) aggressive environmental conditions. Further examples for usage of the invention are Micro-Electro-Mechanic-Systems (MEMS), but also a pressure sensor, blood gas sensor, a glucose meter, such as a blood glucose meter, or the like.

Further fields of concern for the present invention, where the enclosure has been designed to accomplish at least one of these problems, might include providing a medical treatment and/or a medical treatment device for diseases or corporal malfunctions of animals or human beings; or it might comprise a standardized leaking container for certification of product lines; or it might comprise a humidity sensors.

Further fields of usage for the present invention can be found in augmented reality, such as non-fogging waveguide assemblies; or in home appliance/consumer fields such as scent dispensers; or in automotive such as gas sensors. The present invention can also be designed to work as a purification device for water or other liquids, or gases. For example, the invention may also be used in the scope of electro mobility; But also in aviation and space environment, in high temperature environments and in the field of micro optics.

The aforementioned applications concern devices faced with rough environmental conditions and which thus have to be designed especially robust or protected from these conditions—and/or they concern biological interaction with the enclosure.

Further, the invention may, as one option of a solution to the problems, allow to some extent an exchange, such as a fluid exchange, with the surroundings where the enclosure is implemented.

Therefore, and to summarize, it is an object of the invention to provide a means capable of interacting with, changing or measuring properties of a fluid, and ideally small enough to be placed into the same fluid.

In a particular interesting embodiment and to explain one showcase of the invention the possibility to dispense a dosage of a fluid such as a medicament or a chemical substance over time to the surroundings of the enclosure may be provided, such as insulin, e.g. for the treatment of diabetes. The surroundings of the enclosure are hereinafter referred to as outside. In the same embodiment and/or at the same time the blood sugar level could be measured to derive the amount of insulin needed over time. Advantageously, in the same embodiment a depleted enclosure could communicate or send a signal.

The object of the invention is achieved by subject matter of the independent claims. Preferred embodiments of the invention are subject of the dependent claims.

An enclosure according to the invention comprises at least a first substrate and a second substrate, wherein the first substrate is transparent at least in part and/or at least for a wavelength bandwidth. The first substrate may be having an outer surface and the second substrate may also be having an outer surface. The first substrate and the second substrate are typically arranged next to each other in such a way that an inner surface of the first substrate is adjacent to an inner surface of the second substrate. In other words, in this example the two substrates are arranged next to each other, i.e., in direct proximity to one another, wherein the inner surface plane of the first substrate is in contact with the inner surface plane of the second substrate. Typically, each substrate of the enclosure is comprised rather flat, in the meaning that its lateral dimensions are far greater than its width. The outer surface as well as the inner surface opposing the outer surface comprise comparably longer dimensions than the circumferential rim, which lies in between the outer surfaces. By aligning several, at least two, substrates next to or above one another, a stack of substrates is formed. Such a stack of substrates may then be laser-welded in order to firmly join the substrates with each other. For example, for each two substrates, one laser weld line may be used for joining these two substrates. Therefore, for example, in the case that four substrates shall be used to form the enclosure, at least three laser weld lines may be introduced, where each laser weld line is arranged such to join the respectively two neighboring substrates of the substrate stack.

The top most substrate, preferably comprises said transparent material and/or is transparent at least in part of its surface or volume and/or at least for a bandwidth of wavelengths. Whenever the laser is used for welding and joining the substrates to each other, the laser therefore can pass through the top most substrate in order to e.g. reach the interface zone in between the two substrates to be welded. It is referred to the top-most substrate as the laser is typically shot into the substrate(s) from above. It is clear that reference is made to that substrate through which the laser has to pass in order to arrive at the laser spot where it is intended to be positioned. So in a setup where it is feasible to shoot the laser from the side or from below, then the reference to the “top-most substrate” may no longer be applicable, but the “top most substrate” typically refers to the substrate which, during laser welding, is closest to the laser source. In any case, it is preferred, that the substrate material (substrate volume) through which the laser travels in order to place the laser spot inside the substrate/substrate stack, comprises transparent material and/or is transparent at least in part of its surface or volume and/or at least for a bandwidth of wavelengths, typically the bandwidth of the laser used for welding.

The enclosure therefore comprises at least one common laser weld zone wherein material of the first and second substrates is mixed, wherein the laser weld zone is designed such to permanently join the first substrate to the second substrate and/or to provide for a hermetic sealing between the first substrate and the second substrate. Such a at least one laser weld zone extends from within the first substrate to within the second substrate and permanently joins the first substrate to the second substrate. The laser weld zone may further be designed to provide hermetical seal to a function zone situated such that it is surrounded by the laser weld zone. But on the other side, also additional laser weld line(s) may be provided to introduce, assure or improve bonding and/or hermetic seal of a function zone.

For example, the laser weld zone could comprise a first separation spacing S1 to the outer surface of the first substrate and a second separation spacing S2 to the outer surface of the second substrate. If separation spacings S1 and S2 are applied, the laser weld zone may be fully enclosed inside the enclosure. This means, in other words, that the laser weld zone is fully enclosed within the enclosure without touching or penetrating either outer surface of the first or second substrate. So to say, the laser weld zone is fully enclosed with material from either the first substrate or the second substrate, or any substrate comprised in the enclosure. The enclosure therefore fully encloses the laser weld zone, which is not in contact with the environment surrounding the enclosure. This does also mean that neither the first outer surface nor the second outer surface, which is the outer surface of the second substrate, is deteriorated by the at least one laser weld zone. This may increase mechanical and/or chemical resistance of the enclosure. It may also be advantageous if the laser weld zone is initially fully surrounded by material of the enclosure, e.g. including separation spacings S1 and S2, but the enclosure may, e.g. in a later process step, be thinned down so that the laser weld zone is exposed to the surrounding. E.g. the laser weld zone may then also form part of the first outer surface and/or of the second outer surface. Reference is made in this respect, and in particular with respect to methods of polishing down one of the substrates of an enclosure, to German Patent Application DE 10 2020 117 194.3, which is hereby incorporated in its entirety into the present application.

The laser weld zone can be a portion of the first substrate and/or it may be a portion of the second substrate. Preferably, the laser weld zone comprises material from the first substrate as well as material from the second substrate. In other words, material from the first substrate is melted and at the same time material from the second substrate is melted, and the materials are mixed with each other. The laser weld zone may further comprise a refractive index that differs from a refractive index of non-welded portion of the first and/or second substrate.

The enclosure according to the invention further comprises at least one function zone, for example comprising a cavity, enclosed inside the enclosure. Said function zone may be circumferentially enclosed in the enclosure. Such a cavity may further comprise one or more devices or components to be enclosed inside the enclosure. The function zone may also comprise some detector means, some microelectronic or mechanical system and/or any micro optics. Even an energy-generating device like a battery or an energy-harvesting device such as a small solar cell may be provided in the function zone. The function zone can comprise a coating, for example situated in between the two substrates or provided on one of the inner sides of the substrates. Advantageously, the preferred laser welding processes cause only localized heating within the laser weld zone and does not affect the function zone. This allows the use of heat sensitive coatings, such as a coating comprising antibodies in biotechnological applications.

The cavity may, for example, be arranged in the plane of the first and/or second substrate. By way of example, the cavity may be excavated from the first substrate and/or from the second substrate, for example by means of an abrasive method. In the case that three substrates are used to provide the enclosure, the uppermost and lowermost substrate may also be continuous substrates, where the function zone or cavity is arranged in the plane of the intermediate substrate. For example, the intermediate substrate can comprise holes, which become the cavity, when the three substrates are stacked on one another.

The enclosure further comprises at least one fluid channel allowing for a fluid flow between said function zone and an outside. Such a fluid channel allows for direct contact and/or exchange of (parts of) the inside of the enclosure with the surrounding fluid, allowing for interaction of the functional means inside the cavity with properties of the surrounding. For example, it might allow for an inflow of fluid into the cavity and thus for analysis of its properties, and/or it might allow for an outflow of fluid into the surrounding and thus for influencing properties of the surrounding fluid.

The fluid channel has a height (h), wherein the height (h) of the fluid channel is defined by (i) a volume increase of material of the first substrate and/or the second substrate within the laser weld zone and/or (ii) ablation of material of the first substrate and/or the second substrate outside of the laser weld zone and/or selective etching of material of the first substrate and/or the second substrate outside of the laser weld zone.

The volume of the material of the substrate may be changed by changing the phase of the material as the density of a material may change when the materials phase is changed, e.g. from a crystalline phase to an amorphous phase. For example, at least one of the first substrate and the second substrate may be a crystalline material which is selected such that the laser welding process will cause a change of the material phase within the laser weld zone from the crystalline phase to an amorphous phase. When the material is further selected such that the density of the amorphous phase is less than the density of the crystalline phase, this phase change will cause a change in volume such that the volume of the material is increased within the laser weld zone and protrusions of amorphous material are formed within the laser weld zone. These protrusions will cause the two substrates to separate and form a gap which defines the fluid channel. A suitable example of such a crystalline material is sapphire. The height of the fluid channel can easily be adjusted by choosing the size of the laser weld zone. If one laser weld line is insufficient to provide the desired height, two or more laser weld lines may be arranged at different depths of the stack comprising the two substrates. Suitable crystalline substrate materials which change their density when a phase change occurs include, for example, sapphire, quartz, diamond, corundum, glass ceramics, lithium niobate, chalcogenide, and mineral crystals.

Additionally or alternatively, the height of the fluid channel may be adjusted by selective ablation and/or selective etching of the surface of the first substrate and/or the second substrate. Preferably, a laser process is used for ablation of the surface. A particular preferred process for ablation is a combined laser and etching process, wherein in a first step local defects are introduced into the respective substrate by means of a laser, for example in the form of a series of filament-shaped flaws extending into the depth of the substrate, and subsequent etching. A surface of the fluid channel formed by such an ablation process of the first substrate and/or the second substrate has a structure comprising a plurality of contiguous, rounded, dome-shaped depressions. A suitable alation process is, for example, disclosed in U.S. Pat. No. 11,534,754. Preferably, the surfaces formed by the ablation process have an average roughness (Ra) which is below 5 μm, preferably below 3 μm, preferably below 1 μm, and is preferably at least 50 nm. A preferred selective etching may, for example, comprise coating of the surface with a resist, in particular a photoresist, structuring the resist, e.g. by exposing the photoresist and removing of undeveloped parts of the photoresist, and etching. After the etching, any remaining resist may be removed. A resist structure may also be provided by coating the surface with a metal such as gold or chromium, e.g. by sputtering, laser structuring of the coated metal layer and then etching.

In a particular interesting design, the at least one fluid channel may function like a gate that is defined by the quantity of fluid passing through it. In particular, the passage of fluid through the at least one channel might be regulated by a channel regulator.

Preferably, at least one surface of the at least one fluid channel has a functionalized coating. Such a coating may, for example, be applied to an entire surface of a substrate prior to laser welding. Preferred laser welding processes make use of ultra short pulse lasers and cause only localized heating of the substrate materials so that the coating outside of the laser weld zone is not affected by the welding process. Since the entire substrate surface can be functionalized in one process step regardless of the final shape and number of the fluid channel(s), the process is easy to perform and cost effective.

Such a functionalized coating may, for example, comprise an organofunctional siloxane. This organofunctional siloxane may be tailored to suit the specific needs of the application and may, for example, be configured to be hydrophobic or hydrophilic. A suitable example is a trimethoxysilane having the structure R—Si—(O—CH₃)₃, wherein R is a polar ligand in case hydrophobic properties are desired and R is a nonpolar ligand in case hydrophilic properties are desired. Such a functionalized coating will not negatively affect the laser welding process as the coating is decomposed within the laser weld zone such that only the silicate remains on the substrate. Further, the same coatings which are suitable for the function zone may be used as functionalized coating for the at least one fluid channel. In such cases, the entire fluid channel or a certain part of the fluid channel also serves as function zone.

Preferably, the dimensions of the fluid channel, in particular a width and a height, and/or a functionalization of the surfaces of the fluid channel are chosen to adjust capillary forces. Additional parameters for adjusting the capillary forces include the structure of the surfaces of the fluid channel.

In an improvement of the invention, the first substrate can comprise glass, glass ceramics, plastics or crystal such as sapphire. Alternatively or additionally, the second substrate and/or an intermediate layer may comprise glass, glass ceramics, plastics, metal, silicon, or crystal such as sapphire. But also a material such as paper, porous glass, e.g. CoralPor® from Schott AG, could be used e.g. as a filter element as the second substrate and/or an intermediate layer.

Additionally or alternatively, said laser weld zone is arranged in a welding plane such that the welding plane cuts the laser weld zone e.g. centrical.

Said at least one fluid channel may be defined by the laser weld zone, for example by a shape or extension dimension of the laser weld zone. Said fluid channel may, for example, constitute a trench in the laser weld zone.

Said at least one fluid channel may be arranged in the welding plane. Said at least one fluid channel may further comprise a height h in a direction perpendicular to said welding plane, which is smaller or equal to a height HL of the welding zone in a direction perpendicular to its connecting plane. The laser weld zone may also be situated with a height HL1 inside the first substrate and with a height HL2=h−HL1 inside the second substrate. In other words, the complete laser weld line height HL may be situated inside either the first substrate or the second substrate, which means that no additional material or spacing is present in between the first substrate and the second substrate. This indicates that the first substrate is directly bonded and mixed with the second substrate by means of the laser weld process. When the laser weld line is equally reaching into both substrates, HL1=HL2, but, for example, if the laser weld zone and/or laser weld line is shot deeper into the first substrate, then HL1 is greater than HL2. Still, the sum of HL1+HL2 preferably sums up to HL.

The at least one laser weld zone and/or laser weld line may circumference the function zone in a distance DF, where the distance may correspond, for example, to the height HL or less. The distance DF may also correspond to double the height HL or less.

Said at least one fluid channel may further comprise a flat or mostly rectangular shape. Further, said at least one fluid channel may comprise one or several ridge(s). Said at least one fluid channel may also be provided at opposing sides of the enclosure, for example one channel at every side of the enclosure. Said at least one fluid channel can be provided in the form of one or more nanovoid(s).

Said cavity may preferably be interconnected with said at least one fluid channel. Further, a second cavity may be provided, wherein the second cavity is connected to a second fluid channel. The first cavity may be connected to the second cavity by means of a passage. Said passage may preferably comprise a passage diameter and wherein the passage diameter is smaller than the diameter of the fluid channel and/or the second fluid channel.

The at least one channel may comprise said height (h), for example in a direction perpendicular to said welding plane, of 10 nm or more, preferably 25 nm or more, further preferably 50 nm or more, further preferably 100 nm or more, or 300 nm or more, or even 500 nm or more. Said height (h) of the at least one channel may also be, for example in a direction perpendicular to said welding plane, 1000 nm or less, preferably 750 nm or less, further preferably 500 nm or less, further preferably 250 nm or less, or even 100 nm or less. Said heights are in particular preferred for embodiments in which the height of the at least one fluid channel is defined by a change in volume of the material of a substrate within the laser weld zone. If the height of the at least one fluid channel is defined by ablation of material of a substrate, the height of the at least one fluid channel may be chosen to be higher. For example, the height of a fluid channel defined by means of material ablation may be in the range of from 75 nm to 10 μm, preferably in the range of 200 nm to 5 μm and particular preferably in the range of from 500 nm to 1 μm.

The at least one channel may further comprise a width (b), for example in said welding plane, of at least 5 nm or more, further of at least 20 nm or more, preferably of 100 nm or more, further preferably of 1 μm or more, or even of 10 μm or more, or even 100 μm or more. Said width (b) may also be 2 mm or less, further 500 μm or less, preferably 200 μm or less, further preferably 50 μm or less. The flow rate of the channels depends of the fluid and its (temperature dependent) viscosity as well as the wall roughness through the capillary effect; We characterize the channels with the helium leakage test, because of the following reasons:
It is a non-destructive test.
It is an inert gas
It is a reference substance more independent against influencing factors (e.g. atmospheric moisture, molecular interactions).
Its flow rate is less temperature dependent
   We found that channels with helium leakage rate in the following intervals to exhibit beneficial flowing rates also for other fluids and gasses:
a helium leakage rate of 1×10−8 (mbar×liter)/s or more, preferably of 1×10−6 (mbar×liter)/s or more, and/or provides for a fluid flow rate of 5×10−6 (mbar×liter)/s or less, or 1×10−4 (mbar×liter)/s or less.

The properties of the at least one fluid channel may be chosen such that a fluid may pass through said fluid channel in only one direction, thus providing a one-way-valve function. For example, cavities inside the enclosure may define two reservoirs which are connected by a fluid channel. A first reservoir may contain a polar or hydrophilic liquid, and a second reservoir may contain air. For a fluid channel having a hydrophobic functionalized coating, air may travel from the second reservoir to the first reservoir, but no liquid may travel from the first reservoir to the second reservoir. In case of a lipophilic liquid, a lipophobic functionalized coating may be used to provide the one-way valve function. If the enclosure is immersed in a medium such as a liquid, the outside of the enclosure may serve as a reservoir and may be used instead of the first or second reservoir in the given examples. Accordingly, the at least one fluid channel may be configured to provide a one-way-valve function between the outside of the enclosure and a reservoir contained within the enclosure.

The enclosure may, in an improvement of the invention, further comprise a pressure difference generating means, such as an expandable or shrinkable reservoir, an electromechanical pressing means, a chemical potential gradient source or a micro pump.

Further, the enclosure may be comprising an ingrow preventing surface situated next to or around the at least one opening, the ingrow preventing surface, for example, comprising a biologically incompatible material and/or a smoothened or spiked surface, see e.g. detail Ds in FIG. 2. Spikes of a spiked surface may be used as micro structuring, which counteracts the adhesion of cells, especially because spikes can penetrate the cells of bacteria, for example, as a biocidal surface.

The enclosure may also further comprise a fluid property sensor arranged on the outside of the enclosure to be exposed in full fluid contact. Additionally or alternatively, it may further comprise at least one electrical connection from the function zone to the outside of the enclosure.

The at least one laser weld zone may extend from within the first substrate to within the second substrate so that material from the first substrate is mixed into the second substrate and/or material from the second substrate is mixed into the first substrate to permanently and directly join the first substrate to the second substrate. The at least one laser weld zone may further comprise a first separation spacing S1 to the outer surface of the first substrate and a second separation spacing S2 to the outer surface of the second substrate. In the laser weld zone may a convection zone be present, where material from the first substrate is mixed with material from the second substrate. Further, the laser weld zone could be a portion of the first and/or second substrate having a refractive index that differs from a refractive index of a non-welded portion of the first and/or second substrate.

The laser weld zone is preferably arranged around said function zone, for example circumferentially around said function zone. The laser weld zone may also be arranged such, that it comprises a third separation spacing S3 to the at least one channel or opening. At least one functional component may be arranged in said cavity, such as an electronics component, a MEMS or a MOEMS.

An enclosure as depicted hereinabove may be used for example as a sensor element, a wafer level packaged component, a micro lens compound, a micro optical chip, or an LED or laser diode device. Such an enclosure may in an embodiment also be used as a pharma packaging, a pharmaceutical product emitter, or a blood property sensor.

In the scope of the present disclosure there is further an immersible sensor element for measuring fluid properties, comprising at least a first substrate and a second substrate, wherein the first substrate is transparent at least in part and/or at least for a wavelength bandwidth, at least one common laser weld zone wherein material of the first and second substrates is mixed, wherein the laser weld zone is designed such to permanently join the first substrate to the second substrate and/or to provide for a hermetic sealing between the first substrate and the second substrate, at least one function zone, for example comprising a cavity, enclosed inside the enclosure, and comprising a fluid sensor element, and at least one fluid channel allowing for a fluid flow between said function zone and an outside. A fluid property to be measured might include blood pressure or blood glycose level. The fluid also may be, for example liquor, lymphatic fluid, lacrimal fluid, a glandular secret as e.g. insulin, urine or an other body fluid.

Significantly, also extracorporeal analysis of blood sugar concentration may be obtained by analyzing lacrimal fluid with the inventive immersible sensor element providing for an extracorporeal blood glycose sensor which is not invasive to a patient's body.

In the scope of the present disclosure there is further an immersible donator element for changing or adapting fluid properties, comprising at least a first substrate and a second substrate, wherein the first substrate is transparent at least in part and/or at least for a wavelength bandwidth, at least one common laser weld zone wherein material of the first and second substrates is mixed, wherein the laser weld zone is designed such to permanently join the first substrate to the second substrate and/or to provide for a hermetic sealing between the first substrate and the second substrate, at least one function zone, for example comprising a cavity, enclosed inside the enclosure, and comprising a donator fluid reservoir, and at least one fluid channel allowing for a fluid flow between said function zone and an outside. The donator element may be designed for emitting a pharmaceutical product.

Such an immersible donator may further comprise a controllable or regulated valve as a flow regulator for adjusting the amount and/or time of pharmaceutical product to be emitted.

In the scope of the present disclosure there is also an immersible specific filter element for being deployed in a fluid, comprising at least a first substrate and a further substrate, wherein the first substrate is transparent at least in part and/or at least for a wavelength bandwidth, at least one common laser weld zone wherein material of the first and further substrates is mixed, wherein the laser weld zone is designed such to permanently join the first substrate to the further substrate and/or to provide for an at least partially hermetic sealing between the first substrate and the further substrate, at least one function zone comprising a cavity, and at least a first fluid channel allowing for a fluid inflow into said function zone from an outside, and at least a second fluid channel allowing for a fluid outflow into said outside, wherein the second fluid channel is designed such that selectable particles or components of the fluid are retained inside or outside the cavity when said fluid streams through said filter element. The fluid may be, for example, blood, liquor, lymphatic fluid, lacrimal fluid, a glandular secret as e.g. insulin, urine or an other body fluid.

The fluid also may be a fluid pharmaceutical substance.

The respective fluid may contain corpuscular elements as blood cells or other body cells or may contain particles as for instance particles of pharmaceutical substances e.g. particles having a defined solubility allowing for a defined application of sustained release pharmaceuticals.

The laser weld zone can be designed in such a way that it at least partially hermetically seals the function zone in the enclosure. For example, a laser weld line may be drawn without gap around the function zone, so that the first substrate is hermetically joined to the second substrate. The laser weld zone typically is formed by a series of laser dots in a sequence. These laser dots may be placed close enough to each other, so that multiple laser dots overlap spatially. While the first laser pulses generate the absorption zone, absorption of subsequent pulses heat up the absorption zone and enlarge it toward the laser beam. This heat accumulation in combination with the translation of the sample generates a continuous laser weld line. Such a continuous laser weld line comprises hermetically sealing properties, when it is drawn completely around the function zone. However, according to the invention there is at least one fluid channel allowing for a fluid flow between said function zone and an outside of the enclosure. This arrangement comprising hermetically sealing the cavity by means of the laser weld zone and providing a fluid interconnection between the inside or interior of the cavity and the outside or exterior of the enclosure, e.g. by means of a channel, is termed an at least partially hermetically sealed cavity or an at least partially hermetically sealed enclosure. By means of the laser weld line, the substrates are firmly joined with each other in that the laser weld line melts or mixes the material of the first substrate with material of the second substrate in a “convection zone”, in that the laser dot is placed such that the aforementioned mixture of materials is achieved. In other words, the convection zone is characterized in that material from the first substrate is mixed with material from the second substrate in the convection zone.

The laser weld zone may also be arranged such that it comprises a third separation spacing S3 to the circumferential rim of the enclosure. In other words, the laser weld zone leaves a secure spacing to the circumferential rim so that also said rim is not interfered by the laser weld zone. This spacing may be called a “tolerance zone”. Typically, the enclosure is diced out of a wafer or a wafer stack comprising a plurality of arrangements including each an at least first substrate, a second substrate and preferably one or more intermediate substrates, each arranged in a stack, respectively, so that additional spacing in between the laser weld zone and the rim may be advantageous in that the laser weld zone is not interfered upon dicing the enclosure out of the wafer stack.

In the scope of the present disclosure there is also an enclosure configured as a lab-on-a-chip element, wherein the enclosure comprises at least one function zone within the enclosure which is connected to one or more fluid channels. At least one of the fluid channels connects the enclosed function zone to the outside, allowing for a fluidic communication. The lab-on-a-chip element may comprise two or more function zones wherein at least two function zones are in fluidic communication by means of at least one fluid channel.

The function zone may comprise a functionalized coating. Further, the function zone may be configured as a cavity in which analytes and/or living cells may be enclosed.

The geometry of the one or more fluid channel is defined by the arrangement of the laser weld lines. Accordingly, any desired shape within the plane of the substrate can be used, including fluid channels having varying widths or having a meander structure. Such a meander structure may in particular be useful to mix two fluid streams of two fluid channels or to enhance the contact between a fluid and an analyte contained in a function zone. As the laser welding process does not require a fixed photomask-the exact structure of the fluid channel(s) can be easily adjusted and even individual shapes for each device are possible.

The invention is described in more detail and in view of preferred embodiments hereinafter. Reference is made to the attached drawings wherein like numerals have been applied to like or similar components.

### BRIEF DESCRIPTION OF THE FIGURES

It is shown in

FIG. 1 a schematic cross-sectional side view of an enclosure,

FIG. 2 to 4 schematic top views of embodiments of enclosures,

FIGS. 5 to 9 schematic cross-sectional side views of embodiments of enclosures,

FIGS. 10 and 11 an immersible donator element,

FIGS. 12 to 14 an immersible specific filter element, and

FIGS. 15 and 16 a lab-on-a-chip element.

### DETAILED DESCRIPTION OF THE INVENTION

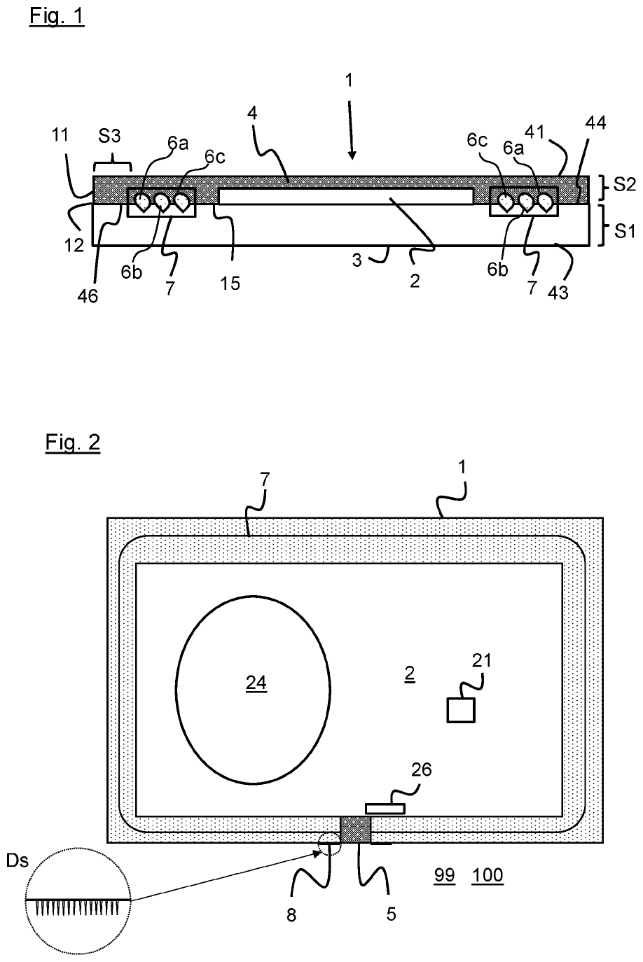

FIG. 1 shows a cross-sectional view of an embodiment of an enclosure 1, where a bottom layer 3 is arranged underneath a top layer 4. The bottom layer or first substrate 3 and the top layer or second substrate 4 are at least partially hermetically bonded to each other by means of three laser weld lines 6a, 6b, 6c forming the laser weld zone 7. The laser weld zone 7 stretches into material of the first substrate 3 as well as into material of the second substrate 4, thus mixing the materials of the two substrates 3, 4. In introducing a firm seal, the laser weld zone 7 may provide an at least partially hermetically sealed area comprising a function zone 2, where one or several laser weld lines 6a, 6b, 6c may be drawn around such an area 2, except for the channel 5. The two substrates 3, 4 contact each other at the inner side 46 of the first substrate 3 and the inner side 44 of the second substrate 4. Both inner sides 44, 46 touch each other at the contact area 15. On the side of the enclosure 1 a spacing S3 is provided in between the edge 11 of the enclosure 1 defining a rim 12 and the laser weld zone 7. In the perspective of FIG. 1, the rim 12 is orientated vertically, whereas the planes 41, 43, 44 and 46 of the first and second substrates 3, 4 are orientated horizontally. The rim 12 may connect the outer side 41 of the second substrate 4 with the outer side 43 of the first substrate 3, so that the total outer surface area may be comprised by the outer side 41 of the second substrate, the rim 12 and the outer side 43 of the first substrate 3.

Referring to FIG. 2, a top perspective view of an enclosure 1 is shown, where laser weld zone 7 is provided partially surrounding function zone 2. A channel 5 interconnects the surrounding outside 99 with the inner side of the function zone 2, e.g. a cavity. So a fluid interconnection between the outside 99 and a cavity inside an enclosure 1 is provided. For example, by means of said channel 5 fluid properties of the outside 99 may be measured, and/or fluid may be donated from the inside to the outside, and/or fluid may be exchanged between the outside 99 and the cavity 2 on the inner side of the enclosure 1. The cavity 2 is preferably hermetically sealed by means of the laser weld zone 7 to all sides except for the channel 5, so that for instance the leakage rate and/or fluid interchangeability can be defined by defining the properties, shape and/or dimensions of channel 5.

Channel 5 may be provided with spikes and other surface properties as additional or alternative ingrow preventing surface 8 already during fabrication, e.g. with laser ablation, which at least counteracts the adhesion of cells. The surface modification can be limited to areas, e.g. entrance areas, so as not to impair the general flow properties.

Further, as depicted in FIG. 2, a reservoir 24 is shown where the reservoir 24 might serve, according to the purpose for the enclosure 1, as e.g. a pressure regulator or as a fluid reservoir for providing a donator fluid to the cavity 2 or via channel 5 to the outside 99. Functional element 21 is shown schematically in the enclosure. A regulating means 26 is also shown schematically which could regulate the inflow or outflow, e.g. by means of a slider for narrowing down the width of channel 5 and such for influencing the possible flow rate through channel 5. Regulating means 26 could also be provided in the form of a soluble barrier which, upon contact with the surrounding fluid at the outside 99, dissolves and allows for fluid exchange. The elements 21, 24, 26 such as the depicted reservoir 24 and regulating means 26 may be provided independently from each other in a cavity 2 of an enclosure 1, but are provided in one common figure for sake of conciseness.

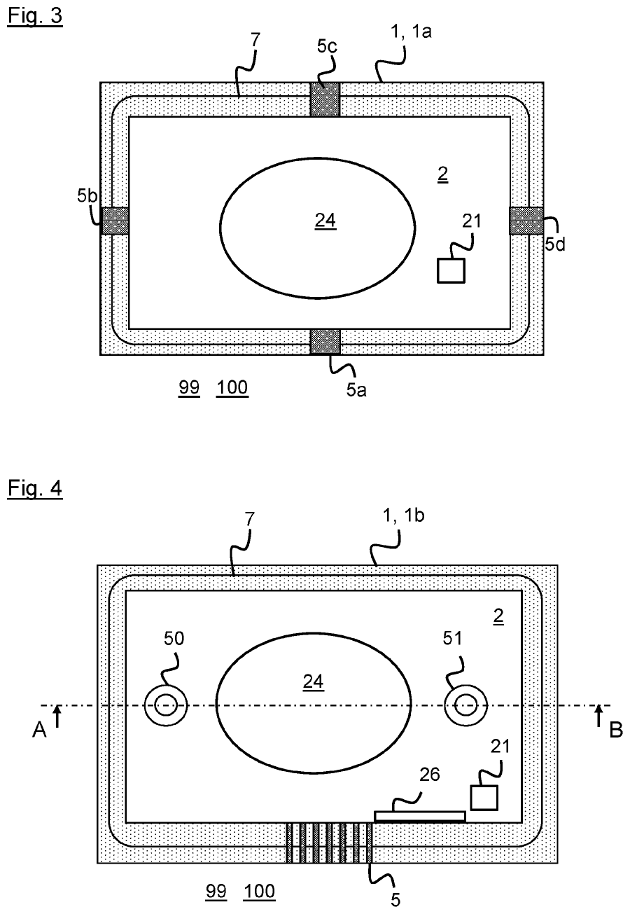

Turning now to FIG. 3 an enclosure 1 is shown with several channels 5a, 5b, 5c, 5d evenly distributed around the rim 12 of the enclosure 1. Again a reservoir 24 and a functional element 21 is shown inside the function zone 2, however depending on the purpose of the enclosure 1 they may be present or not. By providing several channels 5 the leakage rate may be increased. By distributing the channels 5 e.g. on each side of the enclosure 1 an improved fluid flow distribution may be achieved. In particular, the embodiment shown in FIG. 3 can also provide a cross-flow through the enclosure 1 or a fluid exchange, e.g. wherein influx of fluid is allowed via channel 5a, and wherein outflux is allowed via channels 5b, 5c and 5d. Flow rate may be self-regulated or might be regulated by means of functional element 21.

When viewing FIG. 4 channel 5 is provided as several leakage trenches such as micro vias. Such an embodiment may allow for increase of leakage rate in comparison with the embodiment of FIG. 2, but at the same time limit the channel diameter and/or allow for selection of particles that are small enough to pass through channel 5.

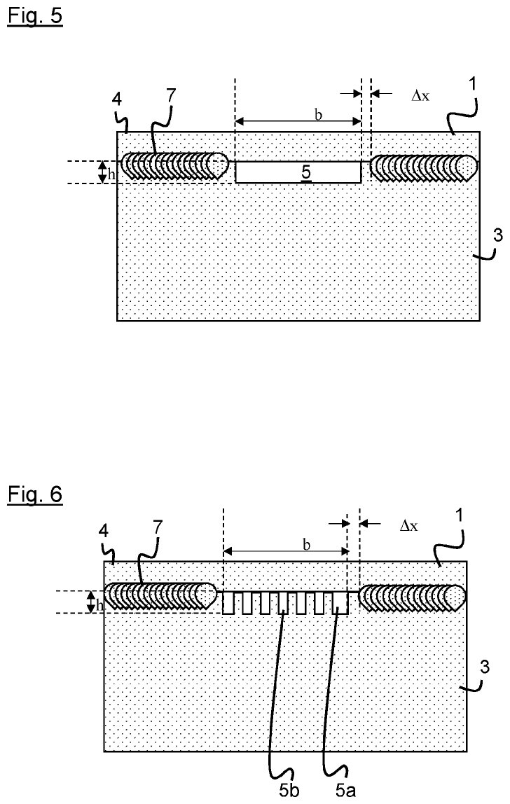

In relation to FIGS. 5 through 9 several designs of channels 5 are discussed with their respective potential making and use. FIG. 5 shows a box-like trench 5 in the plane of the laser weld zone 7, having width b and a lateral spacing Δx. In the making of channel 5 as depicted in FIG. 5 a small shallow trench 5 may be elaborated from the base substrate 3, e.g. by means of ultrashort pulse laser ablation. The height h of the trench may be set in the order of 50 nm up to 1 μm, preferably 300 nm±150 nm. The width b of the trench may be set, by way of example, in the interval between 0 mm up to 2 mm. When both substrates 3, 4 are composed of glass and a glass-to-glass enclosure 1 is used the width may preferably be in between 10 to 20 μm, for example 15±10 μm. Further as an example, b+2 Δx≥w, with w being the width of the laser weld zone 7 (e.g. 40 μm). Herein, Δx is a spacing between the last “shot” of the laser weld line and the beginning of the channel or trench 5. After elaboration of the trench 5 the top substrate 4 may be placed on top of base substrate 3 and contact bonded. The laser weld zone 7 is shot at the interface between top and bottom substrate 3, 4, wherein it stops at a distance Δx before the boundaries of the trench 5 or part-channels 5a, 5b, Since without the trench, the uninterrupted laser weld zone 7 would provide for a hermeticity for the cavity 2, the overall leakage only depends on the properties of the channel 5, like its geometry. Thus an enclosure with a defined leakage rate is provided.

Instead of using laser ablation, the channel 5 may also be elaborated by means of etching, scratching or grinding the rim 12, and/or at least part of the material of one of the substrates 3, 4, 4a may be provided porous, i.e. may comprise a filter material, such as paper, porous glass, e.g. CoralPor® from Schott AG, providing an intrinsic leak rate in the porous material. The channel 5 can be fabricated by a lithography process e.g. in case of silicon. In case of at least one substrate comprising metal or consisting of metal the metal surface may be prepared or provided slightly rough or porous. A polishing pattern may be used which results in a non-flat inner surface of the substrate 3, 4. Also a part of the substrate 3, 4 could be cut out to elaborate the channel 5 in one of the substrates 3, 4.

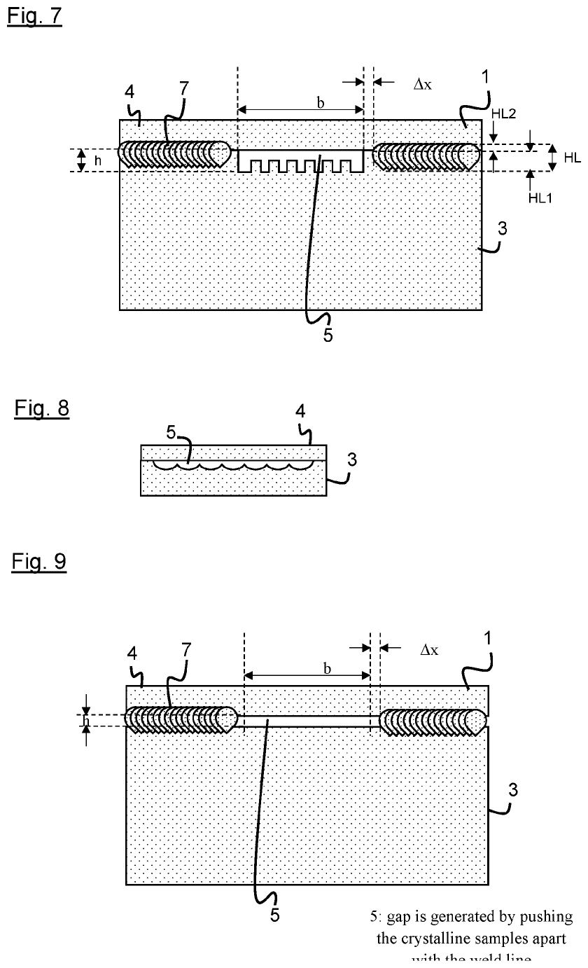

Referring to FIG. 6 a comb-like channel 5 with several part-channels 5a, 5b, . . . is shown, similar to the embodiment shown in FIG. 4. In general, the channel 5 does not have to be homogeneous, it can also be a saw tooth or any other shape with a defined, open cross-section A; it can also be a “lift-off” by the welding lines. FIG. 7 shows another embodiment, where the comb-like structure of the channel 5 is not continuously from bottom to top, but affects only part of the channel 5. An upper part of the channel 5 is shaped continuously, whereas a lower part of the channel 5 is comb-like. FIG. 8a shows a variation where the structure of the channel 5 is shaped by truncations. For example, this form could be obtained by means of drilling.

In yet another example as shown in FIG. 9 an observation obtained from laser welding processes is further developed. During introducing the laser weld zone 7, for example in a sapphire-sapphire-enclosure 1 using crystalline samples, the upper substrate 4 may lift up and separate from the lower substrate 3. The gap that develops in between the substrates 3, 4 can still be hermetically sealed by means of the laser weld. However, if the laser weld zone 7 is not circumferentially closed around the enclosure 1, a gap may persist. When the end points of the laser weld zone 7 are defined such a gap can be embodied as channel 5, where the width can be defined during laser welding by means of the stopping points of the laser weld zone 7.

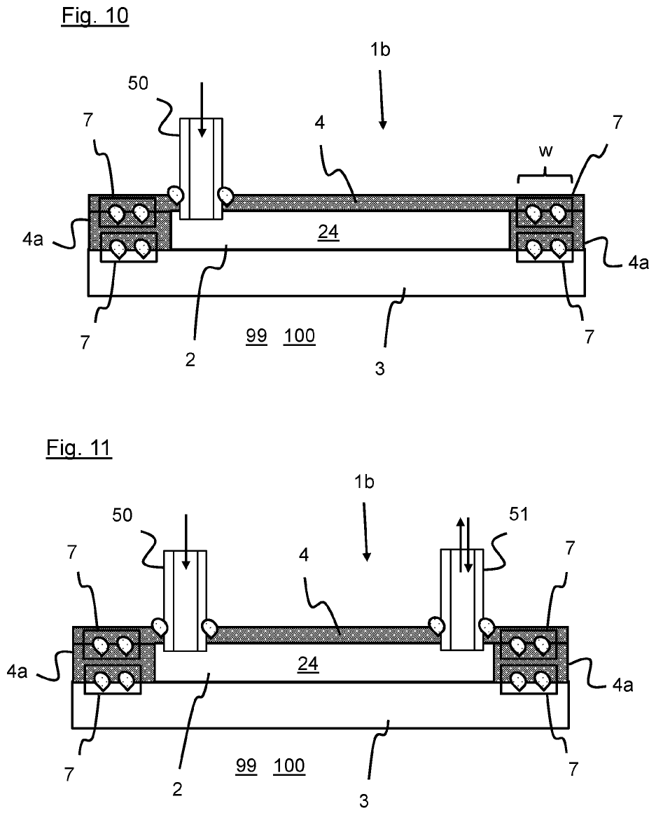

In a further development an enclosure 1 may comprise three substrate layers 3, 4, 4a or more to form the enclosure 1. The first substrate 3 functions as base layer below the cavity 2, the second substrate 4 functions as a top layer above the cavity 2, and an intermediate layer or intermediate substrate 4a is arranged in between those two substrates 3 and 4, see e.g. FIG. 10. The first substrate 3 may thus be welded to the intermediate substrate 4a and the second substrate 4 may be welded also to the intermediate substrate 4a by means of two planes of laser weld zones 7.

In forming the laser weld zone 7 each single shot of a laser pulse generates a nonlinear absorption. This zone may or may not later be visible as an absorptive volume (e.g. black spot). In the latter the term “nonlinear absorption zone” is used to denote the in-situ process volume, where the nonlinear absorption takes place and/or if some permanent modification to the linear absorption has happened, e.g. cases of visible damage in the substrate. This nonlinear absorption zone can cause heat accumulation, that takes place in the direction towards the laser pulse shot. Above the area of nonlinear absorption, which may correspond more or less with the laser focus and which may comprise a size of a few micrometers, an elongated bubble-shaped region may be formed, having only a few micrometers in width but typically up to several tens of micrometer in height (it is also referred to as “bubble” due to its typically quite characteristic shape comparable to an elongated bubble). Around that bubble-shaped region there is situated a melting region, wherein temperatures above Tg may be reached in the glass, which therefore has resolidified (after cooling or dissipation of warmth). The melting region with the included elongated bubble may usually clearly be identified, for example, with a light microscope, since its density and/or the refractive index has changed with respect to the surrounding material of the respective substrate (e.g. glass). In some cases, the area of nonlinear absorption can also be observed as optical damage on the lower tip of the melting region. Each laser spot and thus each laser weld line 6a, 6b, 6c may thus be identifiable, for example by optical means. One single laser spot may also be referred to as heating zone.

An immersible sensor element 1a for measuring fluid properties, such as blood pressure or blood glycose level, may comprise an enclosure 1 as described herein and a sensor element 21, in particular a fluid sensor element 21.

In FIG. 10 and FIG. 11 a cross sectional view in a vertical plane as defined by the arrows A and B of FIG. 4 of an immersible donator element 1b for changing or adapting fluid properties, in particular for emitting a pharmaceutical product or a pharmaceutical substance is shown.

The immersible donator element 1b preferably comprises an enclosure as described herein and at least one function zone in cavity 2 and comprising a donator fluid reservoir 24. In addition, an intermediate substrate 4a may be located between the at least first substrate 3 and second substrate 4 defining cavity 2 by means of a central cut out zone or central opening.

The immersible donator element 1b may further comprise a controllable valve 26 for adjusting the amount and/or time of pharmaceutical product to be emitted to outside 99. Outside 99 may comprise an outside fluid 100 surrounding the immersible donator element 1b.

Port 50 connects cavity 2 with a fluid source not explicitly shown in the drawings which may, when the immersible donator element1b is located within the body of a patient, be an extracorporeal fluid source or may be an intracorporal fluid source for supplying a specific pharmaceutical substance to cavity 2. Due to a pressure in port 50 and the setting of flow regulator 26, the amount of pharmaceutical substance delivered to outside 99 and outside fluid 100 may be controlled in a defined manner.

Significantly, and especially in case of aggressive or toxic pharmaceutical substances, a beneficially defined dilution of the pharmaceutical substance may be obtained before delivered to outside 99. In case of a negative pressure in Port 50, outside fluid 100 may be sucked into cavity 2 and a defined dilution of the pharmaceutical substance and outside fluid 100 is provided in cavity 2. Subsequently, the diluted pharmaceutical substance may be delivered to outside 99 in a defined manner based on an increased pressure in port 50 and a corresponding setting of flow regulator 26. In this embodiment, cavity 2 as a whole may serve as reservoir 24.

In the embodiment shown in FIG. 11, two ports 50, 51 are connected to cavity 2 and may serve in case of port 50 as a fluid delivery and in case of port 51 as a fluid discharge. Then, cavity 2 may be flushed with a cleaning fluid after treatment of the patient's body with the pharmaceutical substance to support an exact timely limited treatment.

If via port 51 a further substance is delivered to cavity 2, this substance may include a further pharmaceutical substance or an activator for the fluid delivered via port 50 and a mixture of pharmaceutical substances and/or an activated pharmaceutical substance may be delivered to outside 99 and outside fluid 100, especially in a defined diluted and timely controlled manner. This embodiment may also assist in the delivery of short-lived therapeutics

These embodiments may be used in general to also reduce a toxic influence on the whole body of a patient by a respective pharmaceutical substance as a very exact and locally confined application is provided, especially in a timely exact defined manner.

It has to be understood that shape and size of ports 50 and 51 are only shown exemplarily and depend on the respective application intended to be realized.

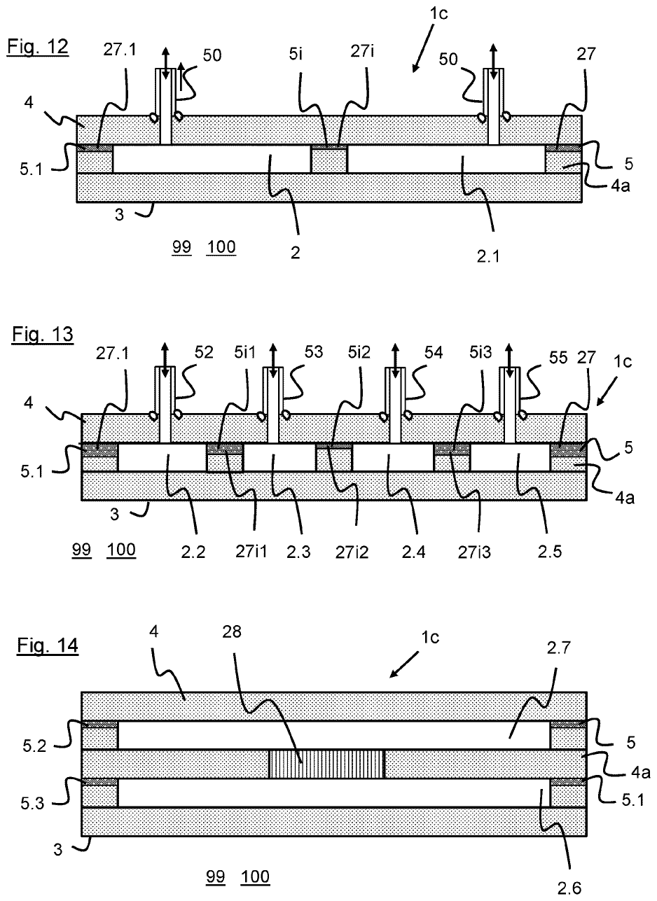

An immersible specific filter element 1c for being deployed in a fluid 99 such as blood as outside fluid 100, is shown in FIGS. 12, 13 and 14 in a cross sectional view extending in a central vertical plane.

The immersible specific filter element comprises at least a first substrate 3 and a further substrate 4, 4a, wherein the first substrate is transparent at least in part and/or at least for a wavelength bandwidth; at least one common laser weld zone 7 wherein material of the first and further substrates is mixed, wherein the laser weld zone is designed such to permanently join the first substrate to the further substrate and/or to provide for an at least partially hermetic sealing between the first substrate and the further substrate; at least one function zone 2 comprising a cavity; at least a first fluid channel 5 allowing for a fluid inflow into said function zone from an outside 99, and at least a second fluid channel 5.1, 5.2, . . . allowing for a fluid outflow into said outside 99, wherein the second fluid channel 5.1, 5.2, . . . is designed such that selectable particles or components of the fluid are retained inside the cavity 2 when said fluid streams through said filter element.

The further substrate may comprise a second substrate 4 or in addition one intermediate substrates 4a or in further embodiments a plurality of intermediate substrates 4a.

Reference is made to FIG. 12, disclosing an immersible specific filter element 1c comprising two cavities 2 and 2.1 defined by respective cut out zones or openings in intermediate layer 4a. Each cavity comprises a channel 5, 5.1 allowing for a fluid flow between function zones in cavities 2, 2.1 and an outside 99, respectively. Outside 99 may comprise an outside fluid 100 surrounding the immersible specific filter element 1c. Between cavities 2 and 2.1 an intrinsic channel 5.i allows for a fluid flow between function zone of cavity 2 and function zone of cavity 2.1.

In a first embodiment, channels 5.1 and 5 comprise each at least a part-channel 5a, 5b . . . , preferably provided in the form of one or more nanovoid(s), having a diameter Da or a height and a width of Da. In this first embodiment, channel 5i comprises at least a part-channel 5a, 5b . . . , which also may be provided in the form of one or more nanovoid(s) based on a later intended use, having a diameter Df or a height and a width of Df with Df<Da.

In a further embodiment, Channels 5, 5.1, 5.i may also comprise each a filter element 27, 27.1 27i having a filter material, such as paper, porous glass, e.g. CoralPor@, having an effective filter diameter in case of channels 5 and 5.1 of Da and in case of channel 5i of Df with Df<Da.

Alternatively, filter element 27 is arranged as a further intermediate layer between

In a further embodiment, via ports 50 a particle containing fluid may be delivered to cavity 2, 2.1 ensuring that particles of a certain size greater than Da remain in cavity 2, 2.1, whereas channel 5i allows for a fluid transport between cavity 2 and 2.1 to allow for a mixture of the fluids delivered via ports 50. If soluble particles will be delivered to captivity 2, 2.1 these particles remain in cavity 2, 2.1 until these particles are resolved to a size smaller than Da ensuring that only a defined maximum concentration of the pharmaceutical substance of these particles is delivered to outside fluid 100.

If a fluid is extracted from cavities 2. 2.1 via ports 50 then it is ensured that only particles having a particle size of less than Da are entering cavities 2, 2.1 as for instance upon a suitable choice of Da red and white blood cells may be excluded from entering cavities 2, 2.1 whereas blood serum and blood platelets may be collected in cavities 2, 2.1 and extracted via ports 50. Erythrocytes typically have a mean diameter in the range of about 7,5 μm and a mean thickness of 2 μm, leukocytes have typically a size of 7 to 20 μm, but blood platelets only a seize in the range of 1 to 4 μm. Accordingly choosing Da in a range from 5 μm to 6 μm may provide for the above mentioned separation. This filtering arrangement allows for a locally restricted and focused real time analysis of respective blood properties of blood without a cumbersome and time-consuming extraction as f.i.by means of syringes or other invasive medical devices.

In FIG. 13 a filter cascade is shown as an embodiment of an immersible specific filter element 1c, comprising four cavities 2.2, 2.3, 2.4 and 2.5 each connected to a respective port 52, 53, 54 and 55. Cavities 2.2, 2.3, 2.4 and 2.5 are defined by cut out zones in intermediate substrate 4a.

In a first embodiment, channels 5.1 and 5 comprise each at least a part-channel 5a, 5b . . ., which also may be provided in the form of one or more nanovoid(s) based on a later intended use, having a diameter Da or a height and a width of Da. In this first embodiment, each channel 5i1, 5i2, 5i3 comprises at least a part-channel 5a, 5b . . ., which may be provided in the form of one or more nanovoid(s) based on a later intended use, having a respective diameter Df1<Da for channel 5i1, Df2<Da for channel 5i2 and Df3 <Da for channel 5i3 or a respective height and a respective width of Df1<Da for channel 5i1, Df2<Da for channel 5i2 and Df3<Da for channel 5i3.

In a further embodiment, Channels 5, 5.1, 5.i1, 5i2, 5i3 may also comprise each a filter element 27, 27.1 27i having a filter material, such as paper, porous glass, e.g. CoralPor@, having an effective filter diameter in case of channels 5 and 5.1 of Da and of Df1<Da for channel 5i1, Df2<Da for channel 5i2 and Df3<Da for channel 5i3.

Based on the size of Df1, Df2 and Df3 complex mixtures of pharmaceuticals comprising soluble particles of a size greater than Df1, Df2 and/or Df3 complex mixtures may be obtained in cavities 2.2, 2.3, 2.4 and 2.5 intermixing of which may be influenced by a positive or negative pressure in ports 52, 53, 54 and 55. A resulting mixture then may be released from cavity 2.5 via channel 5 and from cavity 2.2 via channel 5.1 to the outside fluid 100.

When fluid is extracted from cavities 2.2, 2.3, 2.4, 2.5 via ports 52, 53, 54 and 55, respectively filtered particles with a defined size are contained in these fluids depending on the size of Da, Df1, Df2 and/or Df3 allowing for a defined choice of particle sizes to be extracted.

From FIG. 14 a two chamber embodiment of an immersible specific filter element 1c can be seen comprising two cavities 2.6, 2.7 having in case of cavity 2.7 channels 5 and 5.2 and in case of cavity 2.6 channels 5.1 and 5.3. In this embodiment an array of laser-shot nano-channels 28 is arranged in intermediate substrate 4a allowing for a laterally enhanced fluid exchange between cavities 2.7 and 2.8. The diameter of nano-channels may be chosen to provide a defined filtering effect between cavities 2.7 and 2.8.

Enclosure 1, immersible sensor element 1a, immersible donator element 1b or immersible specific filter element 1c, may be part of a microfluidic arrangement, especially within a complex analytic and/or therapeutic system which may at least in part be implanted into a patient's body for analytic and/or therapeutic purposes. However, the invention is not restricted to medical applications but may also be used in other environments, e.g. chemically harsh environments where materials as disclosed herein provide for an excellent fatigue strength.

Often, during production, a multitude of enclosures 1 comprising a common substrate, a plurality of common substrates or all substrates as common substrates is provided, each having a cavity 2 inside. The enclosures 1 are diced off, which is singulating each enclosure 1. So to say, a multitude of enclosures 1 may be prepared and provided at the same time with the same method step. Method steps of the method of manufacturing an enclosure 1 typically comprise Step A, wherein the substrates are aligned on top of each other, for example as substrates 3, 4, 4a also termed wafers 3, 4, 4a. A to be sealed component 21, 24, 26 may be arranged in Step A in each enclosure 1, if applicable. In Step B the wafers are stacked above each other in order to generate a waferstack, wherein several enclosures 1 will be obtained. The wafers 3, 4a, 4 may be optically aligned on top of each other. In a Step C of the method laser welding each cavity of the wafers 3, 4a, 4 is comprised. Step D could comprise separation or dicing, respectively, of the waferstack and thus singulating the several enclosures out of the waferstack.

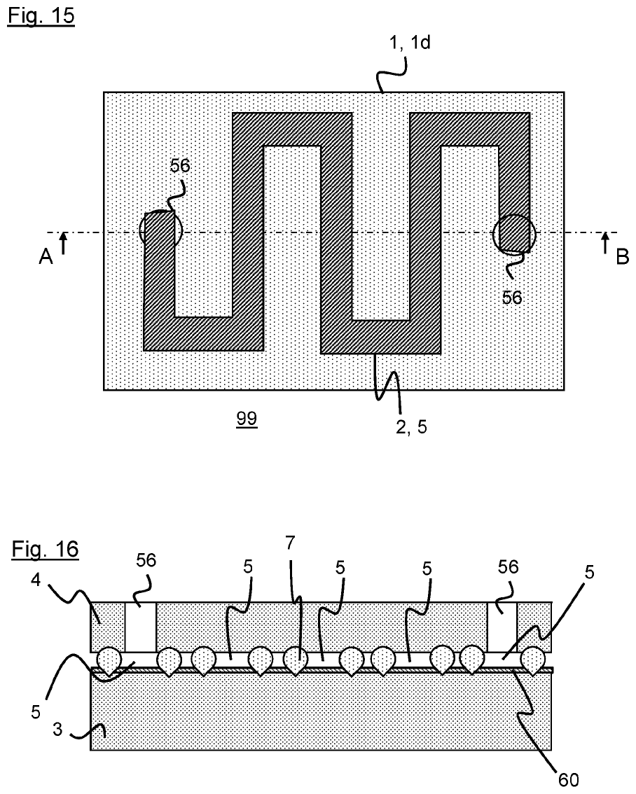

FIGS. 15 and 16 depict an example of an enclosure 1 configures as a lab-on-a chip element 1d, wherein FIG. 15 shows the lab-on-a chip element 1d in a top view. FIG. 16 shows the lab-on-a-chip element 1d in a cross-sectional side view along the line marked A-B in FIG. 15.

The lab-on-a-chip element 1d comprises a first substrate 3 and a second substrate 4 which are stacked on top of each other. A surface of the first substrate 3 is coated with a functional coating 60 on a surface facing towards the second substrate 4. The second substrate 4 has two through holes serving as openings 56. A fluid channel 5 which connects the two openings 56 is defined by laser weld lines 7 and connects the two openings 56. As one of the surfaces of the fluid channel 5 is functionalized with the functionalized coating 60, the fluid channel 5 also serves as a function zone 2. At least one of the substrates 3,4 is chosen from a crystalline material, such as sapphire, so that the introduction of the laser weld lines 7 cause a local increase in volume of the respective substrate's material. Accordingly, the respective substrate 3,4 forms projections within the laser weld zone which lift the two substrates 3, 4 apart. The height of the formed projections defines the height of the fluid channel 5. The shape of the fluid channel 5 is defined by the arrangement of the laser weld lines 7 and can easily be adapted to individual requirements.

A fluid to be processed may be introduced into one of the openings 56. By means of capillary forces, the fluid will be transported through the fluid channel 5 and thus through the function zone 2. The fluid channel 5 has a meandering path so that the fluid to be analyzed will be in close contact with the functionalized coating 60. Depending on the choice of the functionalized coating 60, a reaction between the fluid to be processed and the coating may occur and may, for example, be detected through a color change.

It will be appreciated that the features defined herein in accordance with any aspect of the present invention or in relation to any specific embodiment of the invention may be utilized, either alone or in combination with any other feature or aspect of the invention or embodiment. In particular, the present invention is intended to cover an enclosure 1 configured to include any feature described herein. It will be generally appreciated that any feature disclosed herein may be an essential feature of the invention alone, even if disclosed in combination with other features, irrespective of whether disclosed in the description, the claims and/or the drawings.

It will be further appreciated that the above-described embodiments of the invention have been set forth solely by way of example and illustration of the principles thereof and that further modifications and alterations may be made therein without thereby departing from the scope of the invention.

### Reference List

1 enclosure
1a immersible sensor element for measuring fluid properties
1b immersible donator element for changing or adapting fluid properties
1c immersible specific filter element
1d lab-on-a-chip element
2 function zone or cavity
2.1 second function zone or cavity
2.2 function zone or cavity
2.3 function zone or cavity
2.4 function zone or cavity
2.5 function zone or cavity
2.6 function zone or cavity
2.7 function zone or cavity
3 at least first substrate
4 second substrate
4a further substrate, intermediate substrate or intermediate layer
5 trench/passage defining a channel, especially a fluid channel between the interior of
enclosure 1 or cavity 2 and the outside 99 or exterior of enclosure 1 or cavity 2
5.1 at least a second fluid channel
5.2 at least a second fluid channel
5.3 at least a second fluid channel
5.4 at least a second fluid channel
5a part-channel
5b part-channel
5i intrinsic channel
5i1 intrinsic channel
5i2 intrinsic channel
5i3 intrinsic channel
6a first laser weld line
6b second laser weld line
6c third laser weld line
7 laser weld zone or weld line
8 ingrow preventing surface
11 edge of glass stack/enclosure
12 rim
15 contact area or welding plane
21 functional element
24 pressure difference generating means, reservoir
26 flow regulator
27 filter element
27i filter element
27i1 filter element
27i2 filter element
27i3 filter element
28 laser-shot nano-channels
41 outer side of second substrate
43 outer side of first substrate
44 inner side of second substrate
46 inner side of first substrate
50 Port
51 Port
52 Port
52 Port
54 Port
55 Port
56 opening
60 functionalized coating
99 outside
100 outside fluid
b width of channel 5 in lateral direction perpendicular to the longitudinal extension of channel 5 and welding plane of weld line 7
DF distance of the at least one laser weld zone 7 and/or laser weld line 7 circumferencing the function zone 2 to the function zone 2
HL height of the welding zone in a direction perpendicular to its connecting plane
HL1 height of the laser weld zone HL inside the first substrate
HL2 height of the laser weld zone HL inside the second substrate
h height of channel 5 in a direction perpendicular to said welding plane
W width of the laser weld zone 7
Δx spacing between the last “shot” of the laser weld line and the beginning of the channel or trench 5 in lateral direction perpendicular to the longitudinal extension of channel 5

1. An enclosure comprising:
a first substrate and a second substrate wherein the first substrate is transparent at least in part and/or at least for a wavelength bandwidth;
a common laser weld zone wherein material of the first and second substrates is mixed, and wherein the laser weld zone is designed to permanently join the first substrate to the second substrate and/or to provide for a hermetic sealing between the first substrate and the second substrate;
a function zone optionally comprising a cavity, enclosed inside the enclosure;
a fluid channel allowing for a fluid flow between said function zone and an outside, wherein the fluid channel has a height (h), wherein the height (h) of the fluid channel is defined by
(i) a volume increase of material of the first substrate and/or the second substrate within the laser weld zone and/or
(ii) ablation and/or selective etching of material of the first substrate and/or the second substrate outside of the laser weld zone.

2. The enclosure according to claim 1,
wherein at least one of the first substrate and the second substrate is a crystalline material and is selected such that the material phase within the laser weld zone is changed to an amorphous phase, and wherein the density of the amorphous phase is less than the density of the crystalline phase so that the volume of the material is increased within the laser weld zone and protrusions of amorphous material are formed within the laser weld zone.

3. The enclosure according to claim 1,
wherein a surface of the fluid channel formed by the ablation of the first substrate and/or the second substrate has a structure comprising a plurality of contiguous, rounded, dome-shaped depressions.

4. The enclosure according to claim 1,
wherein the first substrate comprises glass, glass ceramics, plastics or crystal, and/or
wherein the second substrate comprises glass, glass ceramics, plastics, metal, silicon, or crystal, and/or
wherein the second substrate and/or intermediate layer comprises a filter material, and/or
wherein said laser weld zone is arranged in a welding plane such that the welding plane cuts the laser weld zone.

5. The enclosure according to claim 1,
wherein said fluid channel is defined by the laser weld zone, and/or
wherein said fluid channel constitutes a trench in the laser weld zone.

6. The enclosure according to claim 1,
wherein said fluid channel is arranged in the welding plane, and/or
wherein the height (h) of said fluid channel in a direction perpendicular to said welding plane, is smaller or equal to a height of the welding zone, and/or
wherein the laser weld zone is situated with a height HL1 inside the first substrate and with a height HL2=h−HL1 inside the second substrate

7. The enclosure according to claim 1,
wherein said fluid channel comprises a flat or mostly rectangular shape, and/or
wherein said fluid channel comprises one or several ridge(s), and/or
wherein fluid channel is provided at opposing sides of the enclosure, and/or
wherein said fluid channel is provided in the form of one or more nanovoid(s).

8. The enclosure according to claim 1,
wherein the function zone comprises said cavity, and wherein said cavity is connected to said fluid channel, and further comprising
a second cavity, wherein the second cavity is connected to a second fluid channel,
wherein the first cavity is connected to the second cavity by means of a passage.

9. The enclosure according to claim 8,
wherein the passage comprises a passage diameter and wherein the passage diameter is smaller than the diameter of the fluid channel and/or the second fluid channel.

10. The enclosure according to claim 1,
wherein the channel comprises said height (h), of 10 nm or more, and/or
wherein the channel comprises a height (h), of 1000 nm or less, and/or
wherein the channel comprises a width (b), of at least 5 nm or more.

11. The enclosure according to claim 1,
wherein said fluid channel provides for a helium leakage rate of 1×10⁻⁸ (mbar×liter)/s or more, and/or provides for a fluid flow rate of 5×10⁻⁶ (mbar×liter)/s or less, or 1×10⁻⁴ (mbar×liter)/s or less.

12. The enclosure according to claim 1,
further comprising a pressure difference generating means.

13. The enclosure according to claim 1,
further comprising an ingrow preventing surface situated next to or around the opening, the ingrow preventing surface optionally comprising a biologically incompatible material and/or a smoothened and/or spiked surface.

14. The enclosure according to claim 1,
further comprising a fluid property sensor arranged on the outside of the enclosure to be exposed in full fluid contact, and/or
further comprising an electrical connection from the function zone to the outside of the enclosure.

15. The enclosure according to claim 1,
wherein the laser weld zone extends from within the first substrate to within the second substrate so that material from the first substrate is mixed into the second substrate and/or material from the second substrate is mixed into the first substrate to permanently and directly join the first substrate to the second substrate, and/or
wherein the first substrate comprises an outer surface and wherein the second substrate comprises an outer surface and wherein the at least one laser weld zone comprises a first separation spacing S1 to the outer surface of the first substrate and a second separation spacing S2 to the outer surface of the second substrate, and/or
wherein in the laser weld zone a convection zone is present, where material from the first substrate is mixed with material from the second substrate.

16. The enclosure according to claim 1, wherein the laser weld zone is a portion of the first and/or second substrate having a refractive index that differs from a refractive index of a non-welded portion of the first and/or second substrate.

17. The enclosure according to claim 1,
wherein the laser weld zone is arranged around said function zone, and/or
wherein the laser weld zone is arranged such, that it comprises a third separation spacing S3 to the opening, and/or
wherein a functional component is arranged in said function zone.

18. The enclosure according to claim 1, wherein the fluid channel comprises a surface having a functionalized coating.

19. The enclosure according to claim 1, wherein the function zone comprises an antibody and/or the function zone is configured as a cavity comprising living cells.

20. (canceled)

21. An immersible sensor element for measuring fluid properties, comprising:
a first substrate and a second substrate, wherein the first substrate is transparent at least in part and/or at least for a wavelength bandwidth;
a common laser weld zone wherein material of the first and second substrates is mixed, wherein the laser weld zone is designed to permanently join the first substrate to the second substrate and/or to provide for a hermetic sealing between the first substrate and the second substrate;
a function zone for example comprising a cavity, enclosed inside the enclosure, and comprising a sensor element, in particular a fluid sensor element; and
at least one fluid channel allowing for a fluid flow between said function zone and an outside.

22. An immersible donator element for changing or adapting fluid properties, for emitting a pharmaceutical product, comprising an enclosure comprising:
a first substrate and a second substrate, wherein the first substrate is transparent at least in part and/or at least for a wavelength bandwidth;
a common laser weld zone wherein material of the first and second substrates is mixed, wherein the laser weld zone is designed to permanently join the first substrate to the second substrate and/or to provide for a hermetic sealing between the first substrate and the second substrate;
a function zone optionally comprising a cavity, enclosed inside the enclosure, and comprising a donator fluid reservoir; and
a fluid channel allowing for a fluid flow between said function zone and an outside.

23. The immersible donator element of claim 22,
further comprising a controllable valve for adjusting the amount and/or time of pharmaceutical product to be emitted.

24. An immersible specific filter element, for being deployed in a fluid, comprising:
a first substrate and a further substrate, wherein the first substrate is transparent at least in part and/or at least for a wavelength bandwidth;
a common laser weld zone wherein material of the first and further substrates is mixed, wherein the laser weld zone is designed to permanently join the first substrate to the further substrate (4, 4a) and/or to provide for an at least partially hermetic sealing between the first substrate and the further substrate;
a function zone comprising a cavity;
a first fluid channel allowing for a fluid inflow into said function zone from an outside, and at a second fluid channel allowing for a fluid outflow into said fluid, wherein the second fluid channel is designed such that selectable particles or components of the fluid are retained inside the cavity 2 when said fluid streams through said filter element, wherein the first fluid channel has a height and the second fluid channel has a height, wherein the respective height of the first fluid channel and the second fluid channel is defined by
(i) a volume increase of material of the first substrate and/or the further substrate within the laser weld zone and/or
(ii) ablation and/or selective etching of material of the first substrate and/or the further substrate outside of the laser weld zone.
